(19) United States
(12) Patent Application Publication
(10) Pub. No.: US 2012/0209356 A1
(43) Pub. Date: Aug. 16, 2012

(54) EYE PROTECTIVE ARTICLE FOR PERSONAL COSMETIC SKIN TREATMENT

(76) Inventors: Shimon Eckhouse, Haifa (IL); Tuvia Dror Kutscher, Shoham (IL)

(21) Appl. No.: 13/504,126

(22) PCT Filed: Dec. 5, 2010
(86) PCT No.: PCT/IL10/01025

§ 371 Date: Apr. 25, 2012

Related U.S. Application Data
(60) Provisional application No. 61/267,056, filed on Dec. 6, 2009.

(51) Int. Cl. A61N 5/06; A61F 9/04

(52) U.S. Cl. 607/88; 2/15

(57) ABSTRACT

Disclosed is an article designed to attenuate intensive and unwanted light emitted by an IPL or laser device that may occasionally reach the eyes of a user or operator, while not limiting their ability to see in a bright region. Eye protection is achieved by an assembly of baffles extending towards the source of IPL. The baffles limit the field of view of the article to a value sufficient for coupling a safe amount of light intensity enabling comfortable IPL or laser applicator tracking and operation.

### CROSS-REFERENCE TO RELATED APPLICATIONS

This application is being filed under 35 U.S.C. 371 and claims the benefit of the filing date of

United States provisional application for patent that was filed on Dec. 6, 2009 and assigned Ser. No. 61/267,056 by being a national stage filing of International Application Number PCT/IL2010/001025 filed on Dec. 5, 2010, each of which are incorporated herein by reference in their entirety.

### TECHNOLOGY FIELD

In general, the present article relates to light intensity attenuators and, in particular to eye protection articles for personal skin treatment by intense pulse light.

### BACKGROUND

Intense Pulse Light (IPL) and laser light sources are a popular cosmetic and, to some extent, medical skin treatment. IPL delivers broadband light in high intensity pulses. It does not damage the surface of the skin as the intense light is delivered to the deeper layers of the skin, for example, the dermis, leaving the surface layers such as stratum corneum and epidermis practically unaffected. The recovery time is very short and the treated person may return almost immediately to his/her regular activities.

IPL is widely used to improve personal appearance and reduce or eliminate such skin deficiencies as vascular lesions including spider veins, port wine stains, hair removal, facial lines, wrinkles smoothing and others. The procedure is usually conducted by medical practitioners or cosmeticians working under the guidance of a physician. It involves the use of a specially constructed xenon flash lamp and sometimes special optics, usually a lens and a filter to select the light spectrum optimal for the treatment of certain skin areas, for example, a wavelength from 400 nm to 1200 nm or from 590 to 1200 nm. The pulses of light produced by IPL equipment are short in duration, but have a high, eye damaging intensity. Eye protection is worn by the patient and the operator. The patient may use opaque plastic or metal goggles or simply a flexible opaque bandage. The operator may wear special goggles enabling the IPL source observation, although when selecting the skin area to which IPL will be applied may be performed without eye protection.

The simplicity of the IPL treatment has led to development of equipment that may be self-operated by the user in a regular residential environment. The treatment is usually performed in front of a minor with sufficient ambient illumination where the user may visually guide an IPL hand-piece or applicator to a desired location of a skin area to be treated. The user slowly moves the hand-piece over the treatment area as intense pulses of light are delivered. The IPL pulses have an intensity of about 1-20 J/cm2 and occasional reflection or even direct tracking of the applicator location may be unpleasant to the user or even damage the user eye. The risk of eye injury increases when the user treats facial skin and particularly skin segments located close to eyes.

Use of different human eye protection systems such as sun glasses, polarized glasses, and the like, is known, but these systems reduce the ambient illumination and hinder applicator location selection and tracking. In addition these protective glasses are not very effective for IPL light sources due to the broad spectrum of these sources. Laser safety goggles are designed to block impinging light of a certain wavelength and are not applicable in an environment of broad spectrum light, such as that which emanates from the Xenon lamp. The safety of a light-based cosmetic skin treatment device can be increased by incorporation of a sensor enabling protection only when the intense pulse light source is operative. However, use of any type of sensor added to increase eye safety adds complexity and may, of course, fail. Thus, the ideal article designed to protect the eye from intense light and method of such protection should be independent of electronic circuitry.

### GLOSSARY

Several terms are utilized throughout this disclosure. The definition for these terms is provided here for convenience.

The terms “goggles” and “eye mask” as used in the present disclosure have the same meaning and mean an eye protecting article.

As used herein, the term “skin treatment” may include complete or partial hair removal, hair growth retardation, skin rejuvenation, wrinkle reduction, skin tightening, circumferential reduction, vascular lesion removal, texture improvement, cellulite reduction, skin abrasion, acne effects reduction, etc.

### SUMMARY

A passive article that limits the intensity of light reaching the eye of a user in an IPL skin treatment. The eye protection is achieved by an assembly of baffles extending towards the source of IPL. The baffles limit the field of view of the article to a value sufficient for coupling a safe amount of light intensity enabling comfortable IPL or laser applicator tracking and operation. The proportion between the dimensions of the baffles extending from the article towards the source of intensive light determines the amount of light intensity coupled by the article and reaching the eye.

The article may be implemented as a reusable article or as a disposable article.

### BRIEF DESCRIPTION OF THE DRAWINGS

Exemplary embodiments of the eye protective article will be described with reference to the drawings, wherein like numerals represent like parts and wherein:

FIG. 1 is a front perspective simplified view of an exemplary embodiment of the present eye protective article for personal IPL skin treatment.

FIG. 2 is a schematic illustration of a cross section of side perspective view of an eye mask of the present eye protective article for personal IPL skin treatment.

FIG. 3 is a schematic illustration showing determination of the field of view of a baffle.

FIG. 4 is an additional simplified illustration of an exemplary embodiment of baffle structures enhancing light intensity attenuation.

FIG. 5 is a schematic illustration of a cross section of an additional exemplary embodiment of an eye mask of the present eye protective article for personal IPL skin treatment.

### DETAILED DESCRIPTION OF EXEMPLARY EMBODIMENTS

Recently available commercialized personal IPL skin treatment devices enable an inexperienced user to personally conduct a series of skin treatment procedures such as wrinkle smoothing, skin tightening, hair removal, and others. In the absence of qualified personnel during the treatment session, safety of the treatments becomes a major concern. IPL is an intense diffused light that in the absence of proper safety measures may result in user eye injury. Unlike professional IPL devices that treat the target skin location in a static manner, the personal skin treatment devices, for example ‘my elos®” commercially available from Syneron Medical Ltd., Yoqneam, Israel are continuously displaced in course of treatment along the treated skin segment and need to be observed at the time of treatment.

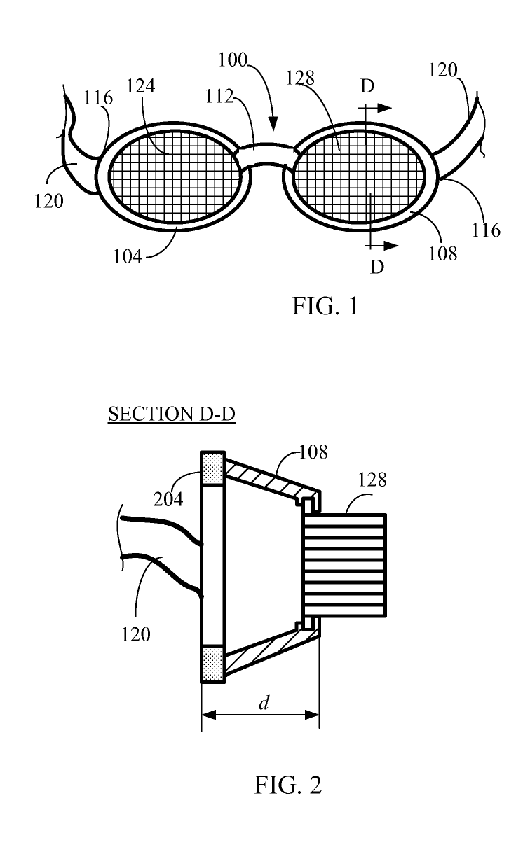

Reference is made to FIG. 1, which is a front perspective simplified view of an exemplary embodiment of the present eye protective article for personal IPL skin treatment. Article or goggles 100 include two eye masks 104 and 108, having a first side facing a light or laser source and a second surface facing the eyes of a user and a centrally located nose bridge 112. Each eye mask has an opening or a buckle (not shown) in outer edge 116 into which a head strap 120 may be inserted and used to secure goggles 100 in place. Both the strap 120 length and the length of the nose bridge 112 are adjustable to fit the size of the user's head. Eye masks 104 and 108 may be made of a non-transparent material such as plastic or other low cost bio-compatible material. An assembly of baffles 124 and 128 is inserted in each of eye masks 104 and 108 respectively, on the first surface facing the light or laser source. Baffles assemblies 124 and 128 are shown as having rectangular or square cross sections, although the baffle cross sections may also be hexagonal, triangular or of any other geometrical shape. Alternatively, baffle assemblies 124 and 128 may be produced as an integral part of the eye masks.

FIG. 2 is a schematic illustration of a cross section of a side perspective view, taken at line D of FIG. 1, of an eye mask of the present eye protective article for personal IPL skin treatment. Opaque silicone or foam gaskets 204 and 208 (not shown) frame eye masks 104 and 108 on the second surface facing, and in contact with, the user's face wherein assemblies 124 and 128 (not shown) baffles extend or are oriented outwards from eye masks 104 and 108 towards the intense pulse light source, the intensity of which is to be attenuated. The gasket allows the goggles to fit snugly across the eyes to eliminate peripheral light or occasional reflected light reaching the user's eye. The depth d of the mask is selected to conveniently accommodate a user's eye.

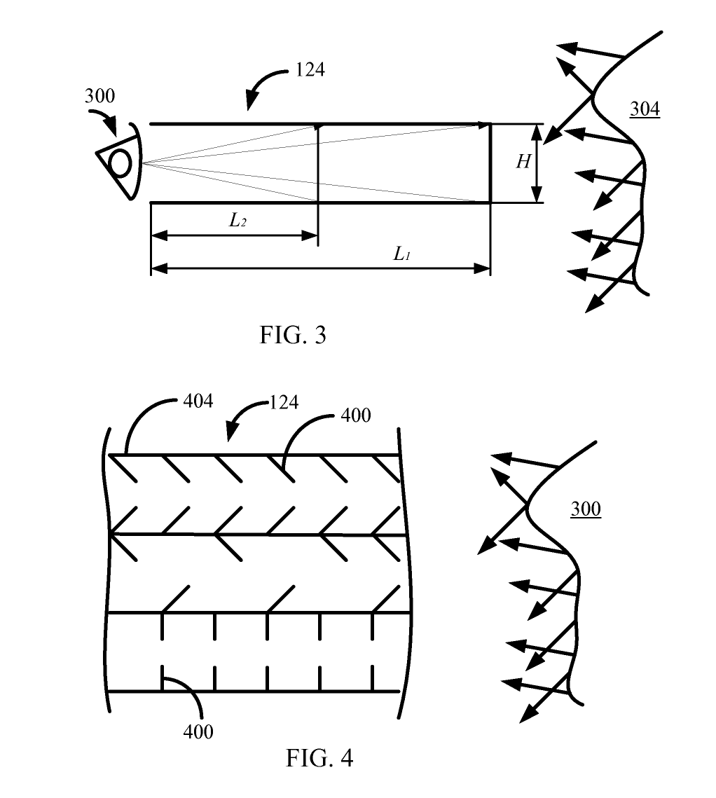

Baffles 124 and 128 reduce the user's field of view. However, without baffles, light from many directions would enter regular goggles and reach the eyes. By using the baffles, most of the stray light can be blocked and prevented from reaching the eye. The amount of light impinging on a baffle is proportional to the field of view enabled by the baffle. FIG. 3 is a schematic illustration showing determination of the field of view of a baffle. The field of view (FOV) can be defined as the angle a user 300 can capture without moving his/her eyes (or head). In case of a square baffle cross section the FOV may be defined as a ratio of H to L. Longer baffles would couple a smaller amount of light or laser intensity and be safer in use. However the selection of a baffle length is governed by the sufficient amount of light intensity entering the eye that enables comfortable applicator tracking. Selection of the baffle dimensions affects also the amount of baffles forming assembly 124 and in some cases one baffle channel may be sufficient. By regulating the H dimension and employing a rectangular instead of a square baffle cross section it is possible to make baffles having different fields of view in vertical and horizontal or other directions optimal for a particular IPL treatment. Numeral 304 marks a source of intense pulsed light (IPL) or laser light.

FIG. 4 is an additional exemplary embodiment of assembly 124 baffles enhancing light intensity attenuation of light originating from light source 300. The attenuating action of the assembly 124 baffles can be further enhanced by adding smaller baffles 400 to the inner space of assembly 124 baffles or providing a light absorbing or dispersing coating. Baffles 400 may be oriented at different angles in respect to the wall 404 of assembly 124 baffles and be of different sizes and shapes.

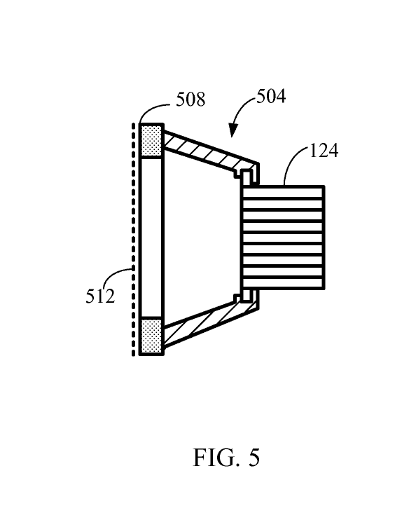

FIG. 5 is a schematic illustration of a cross section of an additional exemplary embodiment of an eye mask of the present eye protective article for personal IPL skin treatment. It is a single eye mask 504 that may be made of a non-transparent material such as plastic or other low cost bio-compatible material. An assembly of baffles 124 is inserted or integrally attached to the side the eye mask 504 facing the light or laser source. Assembly 124 baffles extend towards the source of intense pulse light (not shown). An opaque silicone or a foam gasket 508 frames eye mask 504 on the surface facing, and in contact with, the user's face. Gasket 508 bears an adhesive coating protected by a release layer 512. In use the user removes release layer 512 and adhesively attaches eye mask 504 gasket 508 to the skin immediately surrounding the eyes. The eye mask may be shaped for the right and/or left eyes and provides a protection similar to goggles 100 (FIG. 1). The protruding length of the baffle may be reduced by use of additional baffles similar to baffles 400 (FIG. 4) and locating baffles 400 in the inner space of assembly 124 baffles. The size/footprint of mask 504 is minimal and enables unobstructed access to practically any point on the user face.

For skin treatment the user attaches the goggles or separate eye masks to the skin immediately surrounding the eyes and visually guides the IPL or laser applicator source over any segment of skin to be treated including the face. The gasket allows the eye mask to fit snugly across the eyes and thus eliminates peripheral light or occasional reflected light reaching the user's eyes.

The safety goggles described provide a considerable advantage in protecting the eyes and eyesight of a user from high intensity light while he or she is performing a desired IPL skin treatment. The goggles, at the same time, permit the user to observe the applicator by taking advantage of a portion of the intense light and conduct a skin treatment with a sufficient and comfortable light intensity. Moreover, in addition to providing physiological protection to user eyes, the instant goggles form a unique optical system which may be used by both the user and operator in other skin treatment applications for automatically selecting only a predetermined amount of light and coupling it to a desired optical input.

Several embodiments have been described using detailed descriptions thereof that are provided by way of example and are not intended to be limiting. The described embodiments comprise different features, not all of which are required in all embodiments. Some embodiments utilize only some of the features or possible combinations of the features. Variations of embodiments that are described and embodiments comprising different combinations of features noted in the described embodiments will occur to persons of the art.

What is claimed is:

1. An eye protective article for intense pulse light skin treatment, said article comprising:
a eye mask having a first surface facing a light source and a second surface facing the face of a user; and
one or more baffles terminating at said first surface and operative to attenuate intense pulse light intensity impinging thereon.

2. The eye protective article according to claim 1 wherein said baffles extend from said first surface in a direction away from the face of a user.

3. The eye protective article according to claim 1 wherein said baffles are an insert inserted into the eye mask.

4. The eye protective article according to claim 1 wherein said baffles are an integral part of the eye mask.

5. The eye protective article according to claim I wherein the length and cross section dimensions of the baffles determine the field of view of the article.

6. The eye protective article according to claim 5 wherein the field of view of the article determines the intense pulse light intensity reaching the eyes of a user.

7. The eye protective article according to claim 1 further comprising a soft gasket attached to and framing said second surface of said eye mask.

8. The eye protective goggles according to claim 7 wherein the soft gasket includes an adhesive layer covered by a release material.

9. A disposable eye protective article for personal intense pulse light skin treatment, said article comprising:
a eye mask having one or more baffles terminate at a surface thereof and operative to attenuate intense pulse light intensity reaching the eyes; and
a soft gasket on the opposite surface of said eye mask and framing said surface.

10. A disposable eye protective mask according to claim 9 wherein the soft gasket includes an adhesive layer covered by a release material.

11. A method of intense pulse light skin treatment, said method comprising:
wearing an eye protective mask including one or more baffles;
operating an intense pulse light applicator to treat the desired segment of skin; and
continuously observing and tracking the applicator on skin location.

12. The method according to claim 11, wherein the baffles of the eye protective mask are operative to attenuate the intensity of intense pulse light reaching the eye.

13. A method of intense pulse light skin treatment, said method comprising:
wearing an eye protective mask including one or more baffles operative to attenuate the intensity of intense pulse light reaching the eyes;
operating an intense pulse light applicator to treat the desired segment of skin; and
continuously observing and tracking the applicator on skin location.

14. An eye protective article for intense pulse light skin treatment, said article comprising:
a eye mask having a first surface and a second surface; and
one or more baffles terminating at said first surface and operative to attenuate intense pulse light intensity impinging thereon.

15. The eye protective article according to claim 14 wherein said first surface is facing a light source and said second surface is facing a face of a user.

16. The eye protective article according to claim 14 wherein said baffles extend from said first surface in a direction away from the face of a user.

17. A method of eye protection in an intense pulse light skin treatment procedure, said method comprising:
providing a intense pulse light source and operating the source to perform a skin treatment; and
employing an eye mask to limit treatment provider field of view.

18. An article for eye protection in intense pulse light skin treatment, said article comprising:
an eye mask; and
an assembly limiting the field of view of the eye mask.

19. An article for eye protection in intense pulse light skin treatment, said article comprising:
a eye mask; and
an assembly limiting the angle of light acceptance by the eye mask.

20. The article according to claim 19, wherein the assembly limiting the angle of light acceptance is an assembly of baffles.
